(19) United States
(12) Patent Application Publication
(10) Pub. No.: US 2008/0294050 A1
(43) Pub. Date: Nov. 27, 2008

(54) Ultrasonic Imaging Apparatus

(76) Inventors: Ryuichi Shinomura, Saitama (JP); Kalsunori Asafusa, Chiba (JP); Mitsuhiro Oshiki, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/571,782

(22) PCT Filed: Jul. 6, 2005
(86) PCT No.: PCT/JP05/12456

§ 371 Date: Jan. 8, 2007

(30) Foreign Application Priority Data
Jul. 8, 2004 (JP) 2004-202043

(51) Int. Cl. A61B 8/00

(52) U.S. Cl. 600/459

(57) ABSTRACT

It is possible to realize an ultrasonic imaging apparatus capable of eliminating deterioration of the S/N of the ultrasonic image while suppressing enlargement of the circuit size. The ultrasonic imaging apparatus includes: an ultrasonic probe having a plurality of transducers arranged for transmitting and receiving ultrasonic waves to/from an object to be examined; transmission means for supplying a drive signal to each of the transducers; reception means for phasing/adding and receiving a reflected echo signal received by each transducer; and an image processing unit for reconfiguring an ultrasonic image based on the reflected echo signal received. The transmission means divides the plurality of transducers into a plurality of groups, supplies a common drive signal to the transducers belonging to the same group, and performs focus control by group units.

### TECHNICAL FIELD

The present invention relates to an ultrasonic imaging apparatus and a technique suitable for transmitting/receiving ultrasonic waves to/from an ultrasonic probe in which a plurality of transducers is arrayed.

### BACKGROUND ART

An ultrasonic imaging apparatus radiates ultrasonic waves to an object to be examined from a plurality of transducers arrayed in an ultrasound probe, and constructs an ultrasonic image based on the reflected echo signals generated from the object. In this ultrasonic imaging apparatus, transmission means for performing focus control by providing predetermined delays to drive signals for being provided to the respective transducers of a probe and reception means for receiving the reflected echo signals being outputted from the respective transducers and performing phasing addition are provided. However, transmission means and reception means cause circuit size to be enlarged since each transducer needs a circuit.

In a wave-receiving phasing circuit, adjacent elements are set as one block, and delays between elements within the block as long as long-term delays between the blocks are performed (for example, refer to Patent Document 1 and Patent Document 2). However, a configuration of the transmission circuit is not disclosed in these Patent Documents, therefore reduction of transmission circuit or transmission/reception circuit cannot be achieved.

The objective of the present invention is to provide an ultrasonic imaging apparatus capable of eliminating deterioration of the S/N of the ultrasonic image while suppressing enlargement of the circuit size.

Patent Document 1: JP-1993-256933A

Patent Document 2: U.S. Pat. No. 5,229,933A

### DISCLOSURE OF THE INVENTION

An ultrasonic imaging apparatus comprising:

an ultrasound probe having a plurality of transducers for transmitting and receiving ultrasonic waves to/from an object to be examined;

transmission means for supplying drive signals to the respective transducers;

reception means for phasing/adding and receiving the reflected echo signals received by the each transducer; and

image processing unit for reconstructing an ultrasonic image based on the reflected echo signal received,

wherein the transmission means divides the plurality of transducers into a plurality of groups and supplies a common drive signal to the transducers belonging to the same group.

Here, the transmission means will be described. The transmission means transmits ultrasonic waves from the respective transducers by inputting a common drive signal by the group. Also the transmission means selects all groups or predetermined groups out of the plurality of groups, supplying driving signals to the transducers belonging to the selected groups, and performs focus control by the selected group unit. Alternatively, the transmission means inputs drive signals by thinning out the transducers belonging to the same group, and transmits ultrasonic waves. The bundle units for grouping the plurality of transducers are provided in the chassis of the ultrasound probe.

The transducers are produced by micro fabrication by semiconductor process. Numbers of the transducers belonging to the group to which the common drive signals are inputted increases by the group as they get closer to the center of the bore diameter of ultrasonic waves of the ultrasound probe.

Next, the reception means will be described. The reception means has the first phasing addition means for dividing the plurality of transducers into a plurality of groups and phasing/adding the reflected echo signals being outputted from transducers belonging to the respective groups, and a second phasing addition means for phasing/adding the reflected echo signals being outputted from the first phasing addition means. The first phasing addition means is provided in the chassis of the ultrasound probe.

Number of the transducers belonging to the group wherein the reflected echo signals are performed with phasing addition by the first phasing addition means is different from number of transducers belonging to the group in which the common drive signals are inputted by the transmission means. Number of transducers belonging to the group wherein the reflected echo signals are performed with phasing addition by the first phasing addition means is the same as number of transducers belonging to the group in which the common drive signals are inputted by the transmission means.

Number of transducers belonging to the group wherein the reflected echo signals are performed with phasing addition by the first phasing addition means increases as they get closer to the center of the bore diameter of an ultrasonic wave of the ultrasound probe. The ultrasonic imaging apparatus according to claim 1, characterized in that the bundle unit and the first phasing addition means are constructed in a common circuit. The reception means receives all of the reflected echo signals.

The reception means executes a gradient delay or a focus delay of concave surface, and forms multi-beams. The multi-beam is formed by the first phasing addition means implementing the gradient delay and the second phasing addition means implementing the focus delay. The multi-beam is formed by the first phasing addition means implementing the focus delay and the second phasing addition means implementing the gradient delay.

### BRIEF DESCRIPTION OF THE DIAGRAMS

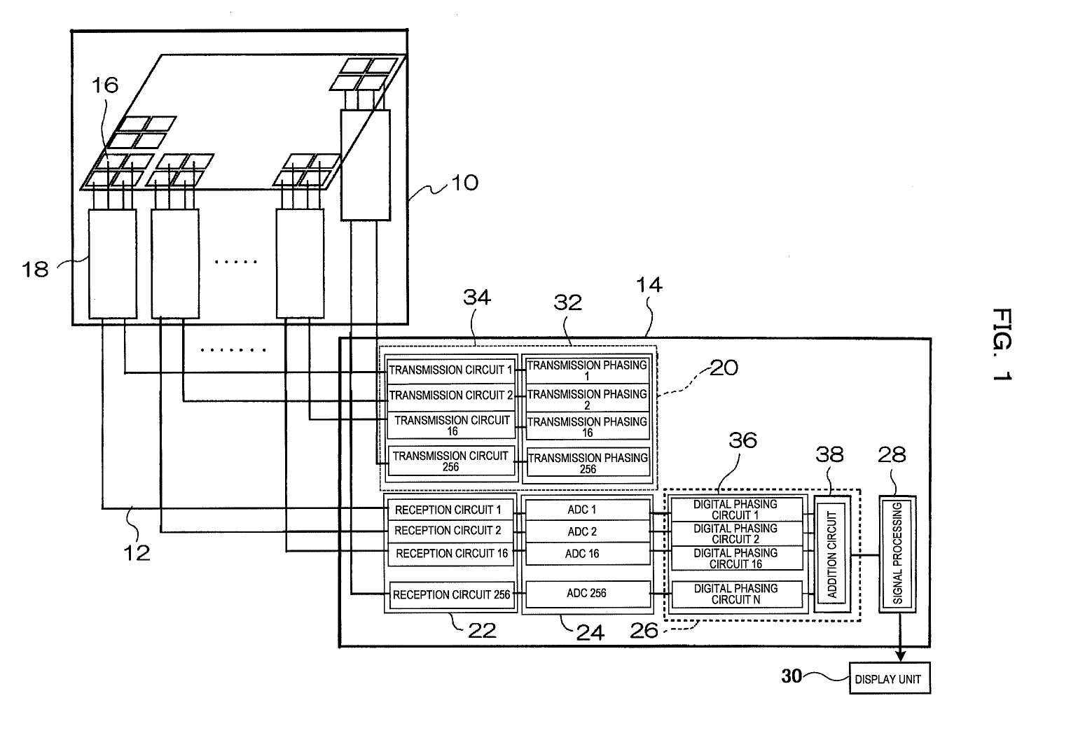

FIG. 1 is a block diagram of an ultrasonic imaging apparatus of an embodiment to which the present invention is applied.

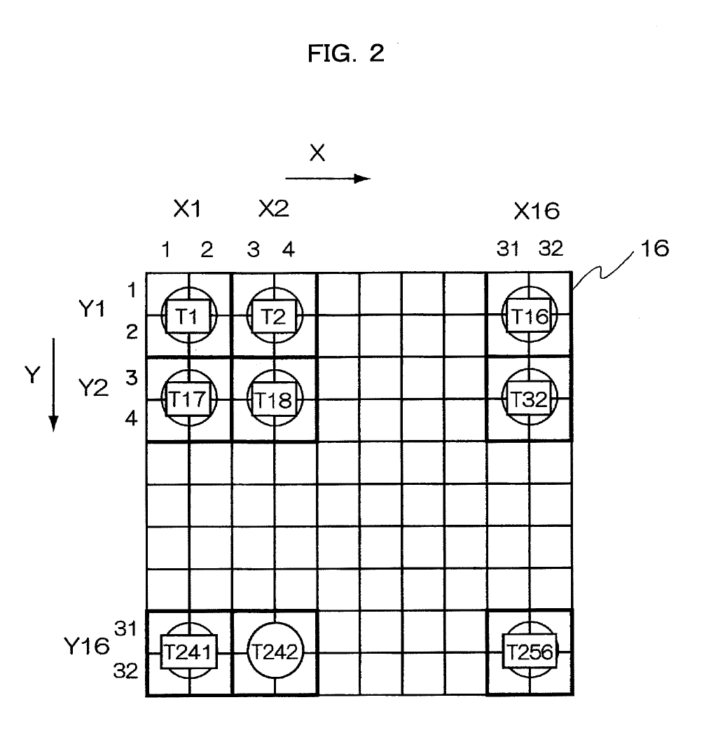

FIG. 2 is a diagram showing the arrangement of the transducers in the ultrasound probe in FIG. 1.

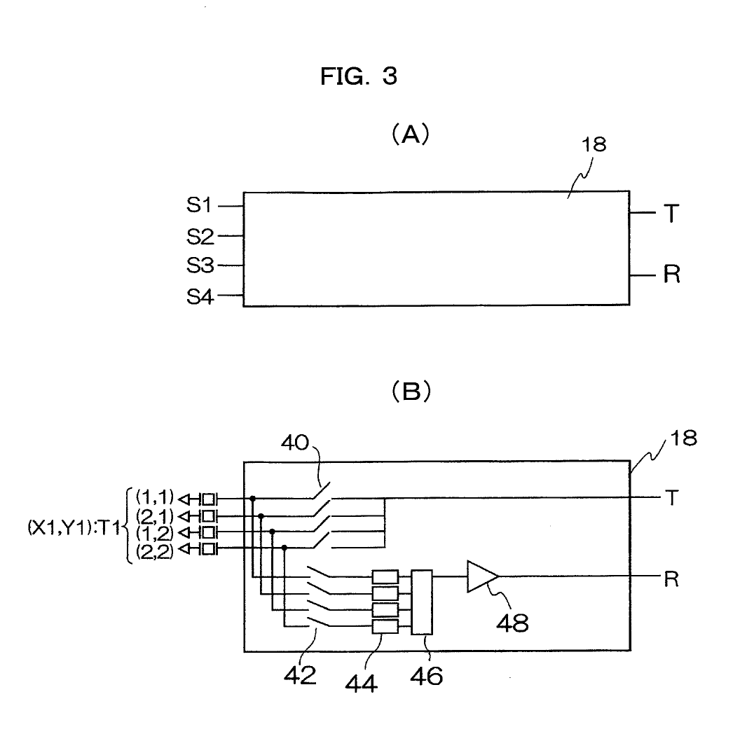

FIG. 3 is a block diagram of a bundle unit in FIG. 1.

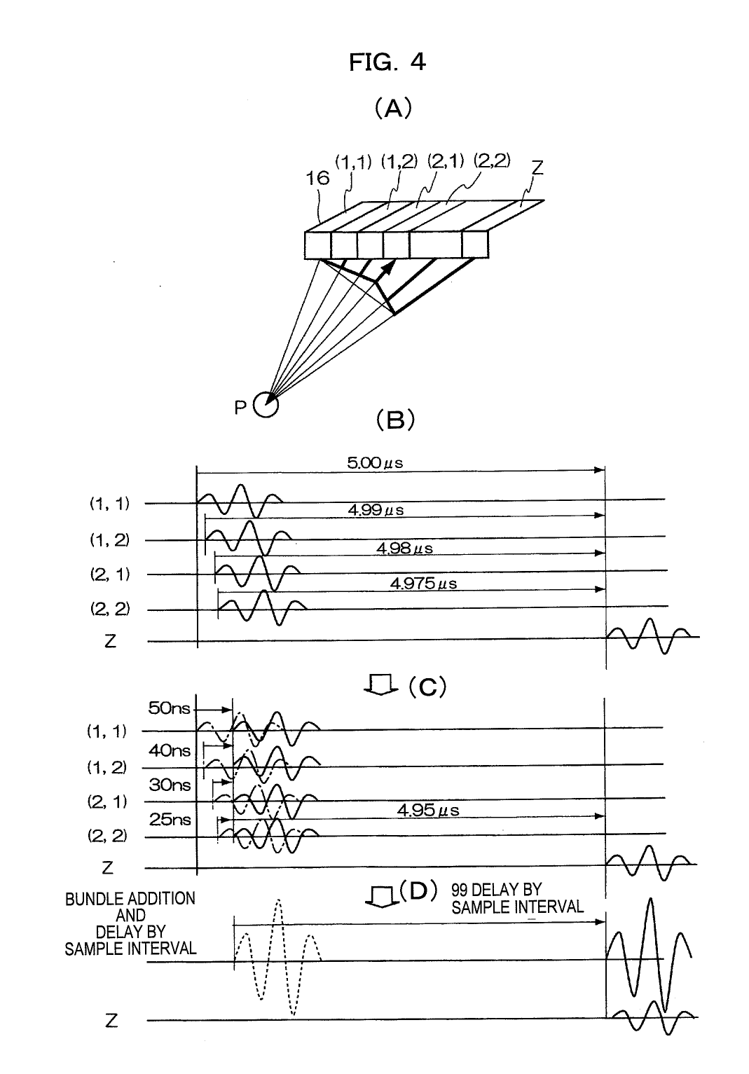

FIG. 4 is a diagram illustrating the reception process of the reflected echo signal in an embodiment to which the present invention is applied.

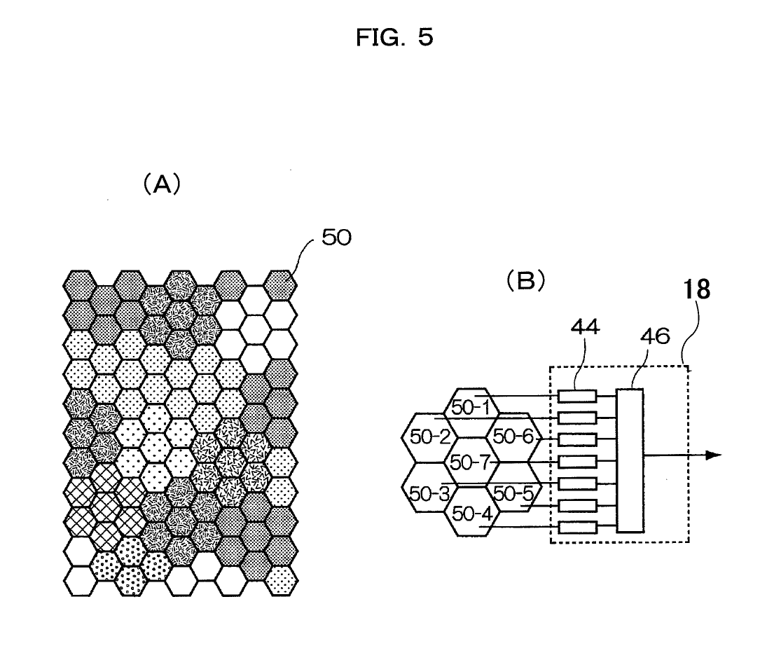

FIG. 5 is another example of an ultrasound probe to which the present invention is applied.

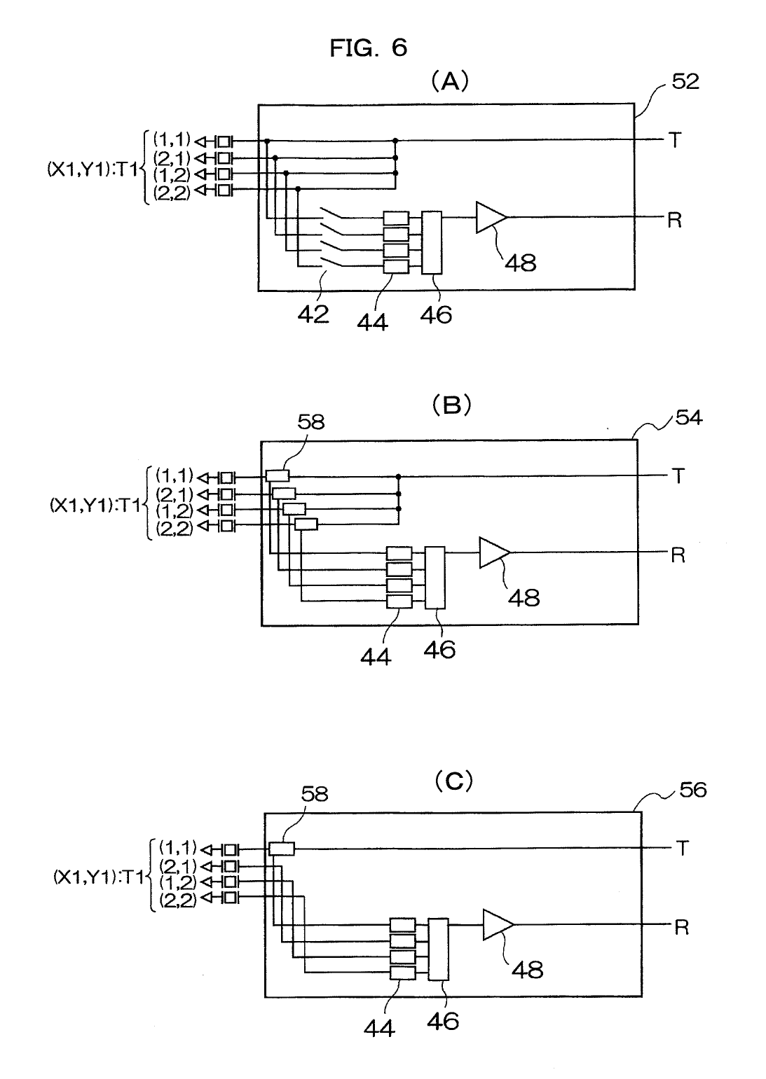

FIG. 6 is another example of the bundle unit.

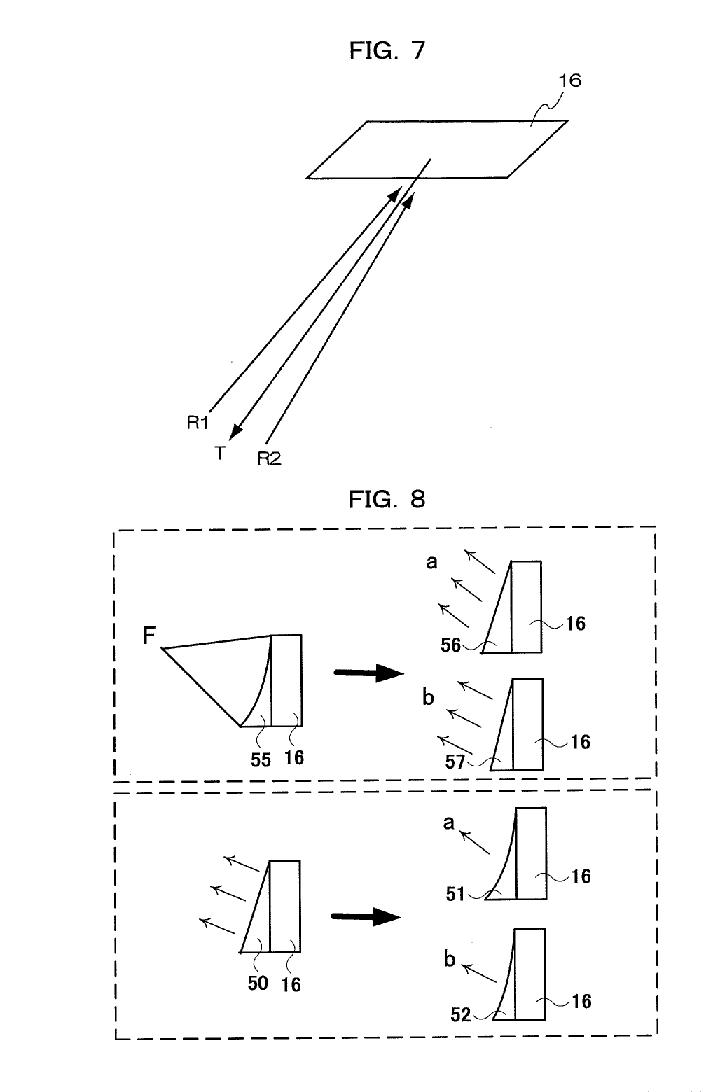

FIG. 7 is a diagram for illustrating a technique for forming a multi-beam.

FIG. 8 is a diagram for illustrating a technique for forming a multi-beam.

### BEST MODE FOR CARRYING OUT THE INVENTION

One embodiment of the ultrasonic imaging apparatus to which the present invention is applied will be described referring to FIGS. 1 ˜4. FIG. 1 is a block diagram of the ultrasonic imaging apparatus to which the present invention is applied. As shown in FIG. 1, the ultrasonic imaging apparatus is configured by ultrasound probe 10 being connected to main unit 14 via a plurality of cables 12.

Ultrasound probe 10 is constructed by 2-dimensionally arraying a plurality of (for example, 1024) transducers 16 for transmitting and receiving ultrasonic beams to/from an object to be examined, and a plurality of transducers 16 is divided into a plural number-n (for example, 256) of groups. Also, n-units of bundle units 18 for supplying a common drive signal to transducers 16 belonging to the same group and performing the first phasing addition by the groups relating to the reflected echo signals outputted from transducers 16 is installed in the chassis of ultrasound probe 10. Each bundle unit 18 is connected to, for example, four transducers belonging to the respective groups via hard wiring.

Main unit 14 comprises:

transmission means 20 for outputting drive signals to the respective bundle units 18;

wave-receiving circuit unit 22 for receiving reflected echo signals being outputted from bundle units 18;

analogue-digital converter unit (hereinafter referred to as ADC unit) 24 for converting reflected echo signals outputted from wave-receiving circuit unit 22 into digital signals according to the control command of the clock unit;

second phasing addition means 26 for phasing/adding reflected echo signals outputted from ADC unit 24; and

signal processing unit 28 as an image processing unit for reconstructing 3-dimensional ultrasonic images based on the reflected echo signals on which phasing addition is performed. The reception means is a generic name including bundle unit 18, wave-receiving circuit unit 22, ADC unit 24, second phasing addition means 26 and signal processing unit 28. Display unit 30 for displaying 3-dimensional ultrasonic images being outputted from signal processing unit 28 and a control unit for outputting control commands to the respective units are also installed.

Transmission means 20 comprises transmission phasing unit 32 for performing focus control on each of the plurality of drive signals by introducing predetermined delay, and transmission circuit unit 34 for outputting the respective drive signals being focus-controlled by transmission phasing unit 32 to the respective bundle units 18. Transmission phasing unit 32 has n-number of wave-receiving circuits, and transmission circuit unit 34 has n-number of transmission circuits. The respective transmission phase circuits of transmission circuit unit 34 are connected to the respective bundle units 18 via simplex cable 12.

Wave-receiving circuit unit 22 is provided with n-number of wave-receiving circuits for receiving reflected echo signals outputted from each of bundle unit 18, and the wave-receiving circuit consists of devices such as a preamplifier and TGC (Time Gain Compensation) circuit for compensating damping of signals in depth direction. ADC unit 24 has n-number of ADC circuits for converting the respective reflected echo signals being outputted from wave-receiving circuit unit 22 into digital signals. The respective wave-receiving circuits of wave-receiving circuit unit 22 are connected to the respective bundle units 18 via simplex cable 12.

Second phasing addition means 26 comprises digital phasing unit 36 for phasing the respective reflected echo signals outputted from ADC unit 24 and addition circuit 38 for adding reflected echo signals outputted from digital phasing unit 36.

FIG. 2 is a diagram showing the arrangement of transducer 16 of the ultrasound probe in FIG. 1. As shown in FIG. 2, 1024 of transducers 16 is arranged in four squares such as 32×32 (32 in X-direction, 32 in Y-direction). Transducers 16 arrayed two-dimensionally are respectively divided in 256 groups of T1˜T256 in the respective squares, so that four transducers arrayed in 2×2 (2 in X-direction, 2 in Y-direction) belong to the same group. Basically, a plurality of transducer 16 are divided into 16 blocks in X-direction as well as 16 blocks in Y-direction, and make 256 of quasi-transducers. For the sake of convenience, arrayal position of transducers is indicated by (x,y). For example, four transducers belonging to group T1 in FIG. 2 are indicated as transducers (1,1), (1,2), (2,1), (2,2). The number of transducers belonging to the same group can be altered, and the blocks may be divided into 9 squares or 16 squares. The blocks may also be a rectangle.

FIG. 3 is a diagram showing a configuration of bundle unit 18 connecting to the respective transducers belonging to group T1 as an example. Bundle units 18 corresponding to other groups are configured in the same manner. As shown in FIG. 3 (A), bundle unit 18 is provided with 2 end-terminals T,R on the side of main unit 14 and four end-terminals S1, S2, S3, S4 on the side of transducers (1,1)˜(2,2). Also, as shown in FIG. 3 (B), end-terminal T is connected to cable 12 as well as diverging into four lines in bundle unit 18, and each of the diverged line is respectively connected to transducers (1,1)˜(2,2) viatransmission switches 40. Transducers (1,1)˜(2,2) are respectively connected to delay circuits 44 via wave-receiving switches 42. Also, addition circuit 46 for adding the reflected echo signals outputted from the respective delay circuits 44 and amplification circuit 48 for amplifying reflected echo signals outputted from addition circuit 46 are installed. Delay circuits 44 and addition circuit 46 are included and referred to as the first phasing addition means.

In such configured bundle units 18, when radiating ultrasonic waves from transducers (1,1)˜(2,2), end-terminal T and transducers (1,1)˜(2,2) are connected by transmission switches 40 being closed as well as wave-receiving switches being opened according to the control command. Also, when they receive ultrasonic waves by transducers (1,1)˜(2,2), transducers (1,1)˜(2,2) and delay circuits 44 are connected by transmission switches 40 being opened as well as wave-receiving switches 42 being opened. In accordance with the above-mentioned control, transmission circuit 34 and wave-receiving circuit 22 are electrically separated, and are protected as a result.

As for delay circuits 44, ones composed of circuits such as analogue sample circuit (for example, CCD, switched capacitor or analogue memory) or LC delay circuit may be used, or ones formed by devices such as ΔΣ modulator may be used. A ΔΣ modulator is composed of devices such as an integration circuit (Σ), quantizer or latch, and is for inputting analogue signals from a simplex input terminal to an integrator, A-D converting the signals outputted from the integrator and outputting them from a simplex output-terminal. Through applying the ΔΣ modulator as delay circuit 44, reflected echo signals can be digitalized in bundle units 18 while enlargement of circuit size is being suppressed.

The operation of such configured ultrasonic imaging apparatus will now be described. First, the side for irradiating ultrasonic waves of ultrasound probe 10 is applied, for example, on a body surface of an object to be examined. Next, according to the input command of an operator, for example, 256 drive signals are generated. To the generated respective drive signals, according to the focus point of an ultrasonic beam being set in advance, predetermined delay is distributed by transmission phasing circuit unit 34. Each of the delayed drive signal is respectively outputted to each bundle unit 18 after processes such as amplification is executed by transmission circuit unit 34. Drive signals inputted to the end-terminal T of the respective bundle units 18 are respectively provided as a common drive signal to the transducer belonging to the respective groups from end-terminals S1˜S4 via transmission switches 40. For example, common drive signal A is provided to transducers (1,1)˜(2,2) belonging to the same group T1. In the same way, drive signal B which has a different phase from drive signal A is provided to the respective transducers belonging to another group (for example, group T2 adjacent to group T1). In other words, ultrasonic waves are transmitted from the respective transducers 16 by common drive signals being inputted by group T1 ˜T256, and transmission beams are formed by these transmitted ultrasonic waves. Through such forming of ultrasonic beams, 3-dimensional ultrasound scan is carried out.

Reflected echo signals generated from an object to be examined are received by the respective transducers of ultrasound probe 10. The received reflected echo signals are outputted to the respective bundle units 18 from the respective transducers 16 by the group unit. The outputted reflected echo signals are amplified after being phased and added by bundle units 18. For example, reflected echo signals outputted from transducers (1,1)˜(2,2) belonging to the same group T1 are respectively inputted to end-terminals S1˜S4 of bundle unit 18. The inputted respective reflected echo signals are performed with phasing by delay circuits 44. The phased reflected echo signals are added by addition circuit 46. The added reflected echo signals are outputted from end-terminal R after being amplified by amplification circuit 48.

The reflected echo signals outputted from bundle unit 18 are converted into digital signals by ADC unit 24 after being implemented with amplification and TGC compensation by wave-receiving circuit unit 22. The digitalized reflected echo signals are added by addition circuit 38 after being phased by digital phasing unit 36. The added reflected echo signals are carried out with various filtering process or signal processing such as envelope-curve processing by signal processing unit 28. Signal processing unit 28 can carry out blood-flow processes such as CFM (Color Flow Mapping) or Doppler processing.

The reflected echo signals outputted from signal processing unit 28 are stored in devices such as a memory as 3-dimensional volume data. The stored volume data are appropriately read out, and 3-dimensional ultrasonic images are reconstructed based on the read-out data. The reconstructed 3-dimensional ultrasonic images are displayed on a monitor of display unit 30 after being converted into signals for display by a digital scan converter (DSC).

As for the transmission of ultrasonic waves, according to the present embodiment, it is possible to reduce the circuit size by providing common drive signals assuming the transducer group (for example, transducers (1,1)˜(2,2)) of the same group (for example, group T1) as one transducer, since it requires provision of the transmission phasing circuits of transmission phasing unit 32 or transmission circuit unit 34 for only the number of these groups (for example, 256).

Also, since ultrasonic waves are received by activating all of the transducers (for example, 1024 transducers), sensibility of the reflected echo signals processed by the reception means is improved and S/N of ultrasonic waves are raised.

For example, in case of an ultrasound probe which is two-dimensionally arrayed (32×32) having 1024 transducers, if the transmission phasing circuit is provided to every transducer, 1024 circuits are necessary which makes the circuit size relatively large. In this respect, the present embodiment requires only 256 transmission phasing circuits thus the circuit size can be reduced.

Next, the reception process of the reflected echo signals will be described referring to FIG. 4. The horizontal axis in FIG. 4 (A)˜(C) are temporal axes. Also as shown in FIG. 4 (A), an example of installing a plurality Z of transducers being horizontally arrayed is used expediently. The transducers (1,1)˜(2,2) in FIG. 4 (A) are corresponding the ones in FIG. 2.

As shown in FIG. 4 (A), distance from focus point P to each of the transducers are respectively different, thus the time that the reflected echo signal generated from point P reaches the respective transducers (hereinafter referred to as arrival time) are also different. Additionally in the present embodiment, sample interval of the sampling clock of ADC unit 24 is set as 50 ns, and the sample interval (delay interval) of the digital sample delay of digital phasing unit 36 is set as 50 ns.

FIG. 4 (B) is a diagram showing the delay time of the reflected echo signals outputted from the respective transducers (1,1)˜(2,2). FIG. 4 (C) is a diagram showing the delay process performed by delay circuit 44. For example, when transducer Z is set as a reference, as shown in FIG. 4 (B), difference between the arrival time of transducer Z and the arrival time of transducer (1,1) is 5.00 μs. Also, delay time of the reflected echo signal of transducer (1,2) is 4.99 μs, delay time of the reflected echo signals of transducer (2,1) is 4.98 μs, and delay time of the reflected echo signal of transducer (2,2) is 4.975 μs.

Here, the time difference between the delay time of the reflected echo signal of the respective transducers (1,1)˜(2,1) and the delay time of the reflected echo signal of transducer (2,2) can be obtained. For example, the time difference between the delay time 5.00 μs of the reflected echo signal of transducer (1,1) and the delay time 4.975 μs of the reflected echo signal of transducer (2,2) can be obtained as 25 ns. In the same manner, the time difference between the delay time 4.99 μs of the reflected echo signal of transducer (1,2) and the delay time 4.975 μs of the reflected echo signal of transducer (2,2) is obtained as 15 ns. Also, the time difference between the delay time 4.98 μs of the reflected echo signal of transducer (1,2) and the delay time 4.975 μs of the reflected echo signal of transducer (2,2) is obtained as 5 ns.

Furthermore, other than the time difference from the delay time of the reflected echo signal of transducer (2,2), micro-delay quantity considering delay interval 50 ns of digital phasing unit 36 (in other words, delay quantity smaller than delay interval 50 ns) can be obtained. For example, remainder of dividing the delay time 4.975 μs of the reflected echo signal of transducer (2,2) by the delay interval 50 ns can be obtained as micro-delay amount 25 ns.

Then the obtained micro-delay amount 25 ns added with the time difference from the delay time of the reflected echo signal of transducer (2,2) turns out to be the delay quantity of the respective reflected echo signals. For example, the reflected echo signal of transducer (1,1) is delayed by 50 ns (time difference 25 ns+micro-delay quantity 25 ns) through delay circuit 44. In the same manner, the reflected echo signal of transducer (1,2) is delayed by 40 ns (time difference 15 ns+micro-delay quantity 25 ns), the reflected echo signal of transducer (2,1) delays by 30 ns (time difference 5 ns+micro-delay quantity 25 ns) and the reflected echo signal of transducer (2,2) delays by 25 ns (time difference 0 ns+micro-delay quantity 25 ns). As a result, the time difference between the delayed respective reflected echo signals and the reflected echo signal of transducer Z turns out to be 4.95 μs. Each of the reflected echo signals delayed in such manner are outputted to wave-receiving circuit unit 22 via amplification circuit 48 after being added in addition circuit 46. In other words, the reflected echo signals received by the respective transducers are bundled by a plurality of bundle units 18 by group T1˜T256. In addition, as for imparting micro-delay quantity 25 ns in relation to the respective reflected echo signals, it is possible to carry it out by mounting the interpolation processing function in digital phasing unit 36 of main unit 14 in place of delay circuit 44.

FIG. 4(D) is a diagram showing the process for performing the phasing by digital phasing unit 36 on the reflected echo signals phased and added by the process of FIG. 4 (C). As shown in FIG. 4(C), the reflected echo signals outputted from transducers (1,1)˜(2,2) are outputted to digital phasing unit 36 via wave-receiving circuit unit 22 and ADC unit 24 after being processed by delay circuit 44 and addition circuit 46 of bundle unit 18. The outputted reflected echo signals are delayed by 4.95 μs (delay interval 50 ns×99 sample) by digital phasing unit 36 as shown in FIG. 4(D). By doing so, the reflected echo signals outputted from transducers (1,1)˜(2,2) are being phased using the reflected echo signals of transducer Z as a reference. In other words, the reflected echo signals outputted from transducer 16 are bundled in the respective bundle units 18 by group units T1˜T256, and the phasing is performed on bundled respective reflected echo signals by digital phasing unit 36.

According to the present invention, since the reflected echo signals are bundled by group units by the first phasing addition means (delay circuit 44 and addition circuit 46 of bundle unit 18), only the number of the groups (for example, 256) of the digital phasing circuits (phasing channels) of digital phasing unit 36 need to be provided, thus it is possible to reduce the circuit size. With the above-mentioned configuration, it is possible to connect two-dimensional arrayed type ultrasound probe 10 to main unit 14 even when main unit 14 is designed for the one-dimensional arrayed type ultrasound probe and provided with fewer phasing channels to use. To sum up, by controlling transmission/reception of transducers 16 of ultrasound probe 10 by group units, the number of the reflected echo signals outputted from ultrasound probe 10 can be aligned with the phasing channels of main unit 14.

For example, in accordance with the present embodiment, in the case of using an ultrasound probe one-dimensionally arrayed with 256 transducers and the main unit is designed with 256 phasing channels, ultrasound probe 10 arrayed with 1024 transducers can be connected to the main unit via 256 cables 12. In this manner, the present embodiment makes it possible to connect an ultrasound probe with a relatively large number of transducers to a main unit having a relatively small number of phasing channels.

Furthermore, in accordance with the present embodiment, since a plurality of bundle units 18 is provided in the chassis of ultrasound probe 10 and the reflected echo signals are outputted to main unit 14 by being bundled by group units by the respective bundle units 18, only the number of groups (for example, 256) of cable 12 connecting ultrasound probe 10 and main unit 14 need to be installed which makes it possible to reduce the amount of hardwiring.

Also, when ultrasonic waves are radiated, they are radiated by, for example, 256 assumed pseudo-transducers, as shown in FIG. 2. On the other hand, when the reflected echo signals are received, they are received by, for example, 1024 transducers. Therefore, since the transmitting-waves and the receiving-waves have different transducer pitch, the generating position of a grating lobe generated due to the transmission of an ultrasonic wave and a grating lobe generated due to the reception of an ultrasonic wave are also different. Accordingly, the increase of grating lobe can be suppressed which leads to the improvement in S/N of ultrasound images. The generating position of a grating lobe can be represented in formula (I). λ represents the wavelength of an ultrasonic wave, θ is the generating position of a grating lobe, θ0 is a scan angle of the beam and pitch represents the pitch width of a transducer.

θ=sin−1(λ/pitch+sin θ0) (1)

While the present invention has been described above based on an embodiment, it is not to be taken by way of limitation. FIG. 5 is another example of the ultrasound probe. As shown in FIG. 5(A), a plurality of transducers 50 having hexagonal-disc-shaped fine structure may be installed hexagonally arrayed in place of the plurality of transducers in FIG. 2. In this case, for example, reflected echo signals outputted from 7 transducer elements 50-1˜50-7 can be bundled by bundle unit 18 as shown in FIG. 5 (B). In such case, 7 of delay circuits 44 need to be provided to bundle unit 18. Also, as transducer element 50, for example, transducer such as cMUT (Capative Micromachined Ultrasonic Transducer: IEEE Trans. Ultrason. Ferroelect. Freq. Contr. Vol. 45 pp. 678-690 May 1998) formed by micro fabrication by semiconductor process can be applied. A cMUT is a micro transducer element wherein the electromechanical coupling factor varies in compliance with the size of impressed voltage. In addition, as for the transducer or mode of transducer element, the one formed by lead zirconate titanate (for example, PZT), multi-layer transducer or the one formed by composite piezoelectric material may be applied.

Also, while an example of applying the present invention to two-dimensionally arrayed type ultrasound probe 10 has been described, it can be applied to the case of using a one-dimensionally arrayed type ultrasound probe. In other words, through applying the present invention when using an ultrasound probe with a relatively large number of transducers, it is possible to construct a high quality image while suppressing the circuit size and compensating the non-uniformity of acoustic velocity.

Also as for ultrasound probe 10, when a plurality of transducers are arrayed to form a rectangular region, there are cases that the beam shapes become different due to the scanning direction of the beam in relation to the side of the rectangular region. Given this factor, a plurality of transducers may be arrayed in a circular region. By such arrangement, since the transducers are arrayed to contact each other in the vicinity of the periphery of the circular region, it is possible to form desirable ultrasound beams by reducing the direction dependency even when the beam scanning is executed in a predetermined direction.

FIGS. 6 (A)˜(C) are other examples of the bundle unit. The difference between bundle unit 52 and bundle unit 16 in FIG. 3(B) is that end-terminal T is directly connected to the respective transducers (1,1)˜(2,2) that belong to the same group T1 by eliminating transmission circuits 40 as shown in FIG. 6 (A). Other configuration is the same as bundle unit 18 in FIG. 3(B). In the present example, wave-receiving switches 42 are opened when ultrasonic waves are radiated from the respective transducers. By such configuration, the same transmission signals are transmitted to the respective transducers (1,1)˜(2,2). Consequently, the size of the circuits can be reduced and circuits such as delay circuits 44 can be protected.

Moreover, as shown in FIG. 6 (B), the difference between bundle unit 54 and bundle unit 52 of FIG. 6(A) is that end-terminal T is connected to the respective transducers (1,1)˜(2,2) via transmission/reception separate circuits 58, as well as the input side of delay circuits 44 is connected to transmission/reception separate circuits 58 by eliminating reception switches 42. In accordance with the present example, drive signals inputted to end-terminal T are provided to the respective transducers by transmission/reception separate circuits 58. Then the reflected echo signals outputted from the respective transducers are inputted to delay circuits 44 by transmission/reception separate circuits 58. By such operation, the reflected echo signals being inputted to the transmission system circuits such as transmission circuits 34 can be avoided, additionally the drive signals being inputted to the reception system circuits such as delay circuits 44 can also be prevented. In other words, by electrically separating the transmission circuits and the reception circuits, it is possible to reduce the load of the transmission system circuits and the reception system circuits.

As shown in FIG. 6(c), the difference in bundle unit 56 from bundle unit 54 of FIG. 6(B) is that end-terminal T is connected only to the transducer (1,1) via transmission/reception separate circuit 58. Here, the transducer (1,1) is connected to delay circuit 44 via transmission/reception separate circuit 58, but other transducers (1,2)˜(2,2) are connected to delay circuits 44 directly. It is an example of sparse-array transducers only in relation to transmission of ultrasonic waves. Through such operation ultrasonic waves are transmitted with predetermined transducers (for example, transducers (1,2)˜(2,2)) being thinned out of the transducers belonging to the same group T1 (for example, transducers (1,1)˜(2,2)). Therefore, reduction of the circuit size can be enhanced furthermore. Also, through using bundle unit 56 properly, it is possible to increase gradually the number of transducers for inputting drive signals as proceeding toward the center of the bore diameter of ultrasonic waves of ultrasound probe 10. It also suppresses the side lobes due to ultrasonic waves transmitted/received to/from the transducers in the vicinity of the edge of the bore diameter of ultrasonic waves. In other words, through adding weight to the transmission of ultrasonic waves, forming of desirable ultrasonic transmission waves can be achieved.

Such addition of weight on the transmission can be carried out by control of the control unit in case of FIG. 3, FIG. 6(A) and FIG. 6(B). In such cases, transmission/reception separate circuits 58 of FIG. 6 (B) need to be provided with the function to block off the drive signals according to the control command. Weighting of the transmission may also be performed by differentiating the amplification of the respective drive signals for inputting to the respective transducers (1,1)˜(2,2). Accordingly, weight can be added to the transmission of ultrasonic waves by group units in any case in FIG. 3 and FIGS. 6 (A)˜(C). Also in the case of FIG. 3 (B) or FIG. 6 (B), weight can be added to the transmission waves of ultrasonic waves even in the same group T1, by turning on/off the respective transducers (1,1)˜(2,2) through controlling transmission switches 40 or transmission/reception separate circuits 58. A buffer circuit or preamplifier may also be installed on the input side or output side of delay circuits 44.

Also, transmission means 20 is capable of selecting all or predetermined groups out of the plurality of groups T1˜T256, providing driving signals to the transducers belonging to the selected groups via bundle unit 18, and performing focus control on them by the selected group units.

The same effect can also be obtained by giving the transmission block and the reception block a separate configuration. Or, making the blocks bigger toward the center may also be effective. The bundle quantity can be increased more toward the center, since the delay difference becomes smaller.

FIG. 7 is a diagram for illustrating a technique for forming a multi-beam. The technique for forming a multi-beam is, as shown in FIG. 7, a technique for forming the reception beam in a plurality of different, for example, two directions of R1 and R2 toward direction T of an ultrasound transmission beam.

For example, multi-beams are formed as shown in the upper level of FIG. 8. An ultrasonic wave is radiated from ultrasound probe 10, and the transmission beam in T-direction is formed by the radiated ultrasonic wave. Then the focus delay is performed on the reflected echo signals being outputted from the respective transducers 16 in delay circuits 44 of bundle unit 18, and dynamic focus is also performed thereon. Or, fixed focus may also be used. Gradient delays 56 and 57 set by digital phasing unit 36 in advance are imparted to the respective reflected echo signals outputted from the respective bundle units 18. The gradient delay is, for example, the delay quantity set in advance for forming the reception beams, and in the present example, it is for forming the two reception beams in the different directions a and b. Through such gradient delay being imparted by digital phasing unit 36 in time division, for example, the reception beams in direction of R1 and R2 are formed approximately at the same time.

Moreover, as shown in the lower level of FIG. 8, gradient delay 50 in the transmission direction is imparted and bundled in the delay circuit of bundle unit 18, and focus delays 51 and 52 are implemented dynamically in respective directions a and b in digital phasing unit 36 of the main unit. It can be implemented either by time-division processing or parallel processing.

By applying such technique for forming multi-beams, a plurality of reception beams can be formed by one transmission beam, thus the ultrasound imaging time can be shortened. Also, in place of imparting the gradient delays in relation to the respective reflected echo signals in time division by digital phasing unit 36, the wave-receiving phasing unit for direction R1 and the wave-receiving phasing unit for direction R2 may be provided in parallel. Moreover, the direction of the wave-receiving beams formed by digital phasing unit 36 is not limited within the two-dimensional plane including direction T1 of the transmission beam, and a plurality of them can be formed in isotropic directions around direction T1. Accordingly, multi-beams can be formed even when ultrasound scanning is carried out three-dimensionally using two-dimensionally arrayed ultrasound probe 10. When ΔΣ modulators are used as delay circuits 44, multi-beams can be formed by executing time-division control on the respective ΔΣ modulators.

Also while the number of the transducers belonging to the group to which the common drive signals are inputted by the transmission means and the number of transducers belonging to the group wherein the reflected echo signals are bundled by bundle unit 18 are different, they may be set to be the same. By such setting, the circuit size can be properly reduced while considering S/N necessary for the ultrasound images in compliance with the imaging regions.

1. An ultrasonic imaging apparatus comprising:
an ultrasound probe having a plurality of transducers arrayed for transmitting/receiving ultrasonic waves to/from an object to be examined;
transmission means for supplying a drive signal to each of the transducers;
reception means for phasing/adding and receiving a reflected echo signal received by each transducer; and
an image processing unit for reconstructing an ultrasonic image based on the reflected echo signal received,
wherein the transmission means divides the plurality of transducers into a plurality of groups and supplies a common drive signal to the transducers belonging to the same group.

2. The ultrasonic imaging apparatus according to claim 1, wherein the transmission means causes ultrasonic waves to be transmitted from the respective transducers by inputting the common drive signal by the respective groups.

3. The ultrasonic imaging apparatus according to claim 1, wherein the transmission means selects all of or predetermined groups from the plurality of groups, provides a drive signal to transducers belonging to the selected groups and performs focus-control by the selected group units.

4. The ultrasonic imaging apparatus according to claim 1, wherein the transmission means transmits ultrasonic waves as thinning out the transducers belonging to the same group and inputting a drive signal to the thinned out transducers.

5. The ultrasonic imaging apparatus according to claim 1, 2, 3 or 4, wherein the bundle unit for dividing the plurality of transducers into groups is provided in a chassis of the ultrasound probe.

6. The ultrasonic imaging apparatus according to claim 1, wherein the transducer is formed by micro fabrication by superconductor processing.

7. The ultrasonic imaging apparatus according to claim 1, wherein the number of transducers belonging to the group to which the common drive signal is inputted by the transmission means increases by the group as proceeding toward the center of bore diameter of ultrasonic wave of the ultrasound probe.

8. The ultrasonic imaging apparatus according to claim 1, wherein the reception means includes the first phasing addition means for dividing the plurality of transducers into a plurality of groups and performs phasing addition on the reflected echo signals being outputted from transducers belonging to the respective groups, and the second phasing addition means for performing phasing addition on the respective echo signals outputted from the first phasing addition means.

9. The ultrasonic imaging apparatus according to claim 8, wherein the first phasing addition means is provided in the chassis of the ultrasound probe.

10. The ultrasonic imaging apparatus according to claim 8 or 9, wherein the number of transducers belonging to the group wherein phasing addition is performed on the reflected echo signals by the first phasing addition means is different from number of transducers belonging to the group to which the common drive signals are inputted by the transmission means.

11. The ultrasonic imaging apparatus according to claim 8 or 9, wherein the number of transducers belonging to the group wherein phasing addition is performed on the reflected echo signals by the first phasing addition means is the same as the number of transducers belonging to the group to which the common drive signals are inputted by the transmission means.

12. The ultrasonic imaging apparatus according to claim 8, wherein the number of transducers belonging to the group wherein phasing addition is performed on the reflected echo signals by the first phasing addition means increases by group as proceeding toward the center of the bore diameter of an ultrasonic wave of the ultrasound probe.

13. The ultrasonic imaging apparatus according to claim 5, wherein the bundle unit and the first phasing addition means are constructed in a common circuit.

14. The ultrasonic imaging apparatus according to claim 1, 2, 3 or 4, wherein the reception means receives all of the reflected echo signals.

15. The ultrasonic imaging apparatus according to claim 1, wherein the reception means forms a multi-beam by executing a gradient delay and a focus delay.

16. The ultrasonic imaging apparatus according to claim 8, wherein a multi-beam is formed by the first phasing addition means implementing a gradient delay and the second phasing addition means implementing a focus delay.

17. The ultrasonic imaging apparatus according to claim 8, wherein a multi-beam is formed by the first phasing addition means implementing a focus delay and the second phasing addition means implementing a gradient delay.

18. The ultrasonic imaging apparatus according to claim 9, wherein the bundle unit and the first phasing addition means are constructed in a common circuit.
